(19) United States
(12) Patent Application Publication
(10) Pub. No.: US 2025/0002955 A1
(43) Pub. Date: Jan. 2, 2025

(54) Method for Promoting Production of Exopolysaccharide by Bifidobacterium Bacteria

(71) Applicant: TOHOKU UNIVERSITY, Miyagi (JP)

(72) Inventors: Ayaka Tamura, Miyagi (JP); Wakako Ohtsubo, Miyagi (JP); Haruki Kitazawa, Miyagi (JP); Binghui Zhou, Miyagi (JP); Yuki Arikawa, Miyagi (JP)

(21) Appl. No.: 18/692,976

(22) PCT Filed: Sep. 20, 2022
(86) PCT No.: PCT/JP2022/034944

§ 371 Date: Mar. 18, 2024

(30) Foreign Application Priority Data
Sep. 21, 2021 (JP) 2021-153486

(51) Int. Cl. C12P 19/04

(52) U.S. Cl. CPC C12P 19/04

(57) ABSTRACT

It is to provide, for example: an exopolysaccharide (EPS) production method with improved EPS productivity; EPS produced by the production method; a method for producing a fermented product comprising EPS, the method having improved EPS productivity; a fermented product produced by the production method; an agent for promoting production of EPS of bacteria of the genus Bifidobacterium; and a method for promoting production of an exopolysaccharide (EPS) of a bacterium of the genus Bifidobacterium. The bacterium of the genus Bifidobacterium is cultured in a culture medium comprising L-fucose.

### TECHNICAL FIELD

The present invention pertains to providing, for example, an exopolysaccharide (EPS) production method with improved EPS productivity, EPS produced by the production method, a method for producing a fermented product comprising EPS, the method having improved EPS productivity, a fermented product produced by the production method, an agent for promoting production of EPS of bacteria of the genus Bifidobacterium, and a method for promoting production of EPS of a bacterium of the genus Bifidobacterium.

### BACKGROUND ART

Exopolysaccharide (EPS) is a generic term for polysaccharides secreted by bacteria to protect themselves from exogenous stress and the like. An EPS is a high molecular weight polymer comprising sugar residues, is mainly composed of polysaccharides (exopolysaccharides) and proteins, and also comprises macromolecules such as DNA and lipids. EPS produced by bacteria such as lactic acid bacteria are known to have functions such as antibacterial properties, water retentivity, osmotic pressure resistance, and immunomodulatory effects. In addition, when a human or the like ingests EPS, effects such as an action of promoting the growth of enteric bacteria and an immunostimulatory effect are obtained, and therefore, in recent years, such EPS have attracted attention for use in functional foods and the like. For example, Patent Document 1 indicates that EPS produced by Lactobacillus delbrueckii subsp. bulgaricus, which is a lactic acid bacterium, can regulate immune balance in a subject such as a human inoculated with the EPS, and Patent Document 2 discloses a method for producing EPS by culturing Bifidobacterium longum, and indicates that such EPS has moisturizing and immunostimulatory effects.

In addition, Patent Document 3 for example discloses, as a method for more efficiently obtaining such EPS, a method in which formic acid is added to a milk raw material culture medium to improve the number of viable EPS-producing lactic acid bacteria, and thereby increase the amount of EPS produced. In addition, Non-Patent Literature 1 indicates that EPS production by Bifidobacterium animalis subsp. lactis is promoted by the use of bile.

However, it was not previously known that EPS production by bacteria of the genus Bifidobacterium is promoted by L-fucose.

### CITATION LIST

### Patent Literature

Patent Document 1: JP 2021-101645 A
Patent Document 2: JP 5192808 B

### Non-Patent Literature

Non-Patent Literature 1: APPLIED AND ENVIRONMENTAL MICROBIOLOGY (2009), Vol. 75, No. 4, pp. 1204 to 1207

### SUMMARY OF INVENTION

### Technical Problem

The present invention addresses the problem of providing, for example: an exopolysaccharide (EPS) production method with improved EPS productivity; EPS produced by the production method; a method for producing a fermented product comprising EPS, the method having improved EPS productivity; a fermented product produced by the production method; an agent for promoting production of EPS of bacteria of the genus Bifidobacterium; and a method for promoting production of an exopolysaccharide (EPS) of a bacterium of the genus Bifidobacterium.

### Solution to Problem

As a result of intensive studies to solve the problem of the present invention, the present inventors discovered that the productivity of EPS using a bacterium of the genus Bifidobacterium is improved by culturing the bacterium of the genus Bifidobacterium in a culture medium comprising L-fucose, and thereby the inventors arrived at the present invention.

That is, the present invention relates to, for example,
(1) a method for producing an exopolysaccharide (EPS), the method comprising a step A of culturing a bacterium of the genus Bifidobacterium in a culture medium comprising L-fucose;
(2) the method according to (1), wherein the bacterium of the genus Bifidobacterium is Bifidobacterium breve or Bifidobacterium lactis;
(3) an EPS produced by the production method according to (1) or (2);
(4) a method for producing a fermented product comprising EPS, the method comprising a step a of culturing a bacterium of the genus Bifidobacterium in a fermented product raw material comprising L-fucose;
(5) the production method according to (4), wherein the bacterium of the genus Bifidobacterium is Bifidobacterium breve or Bifidobacterium lactis;
(6) the production method according to (4) or (5), wherein the fermented product raw material is a fermented dairy product raw material, and the fermented product is a fermented dairy product;
(7) a fermented product produced by the production method according to any one of (4) to (6);
(8) the fermented product according to (7), wherein the fermented product is a fermented dairy product;
(9) an agent for promoting production of an exopolysaccharide (EPS) of bacteria of the genus Bifidobacterium, the agent comprising L-fucose as an active ingredient; and
(10) a method for promoting production of an exopolysaccharide (EPS) of a bacterium of the genus Bifidobacterium, the method comprising a step A of culturing the bacterium of the genus Bifidobacterium in a culture medium comprising L-fucose.

### Advantageous Effects of Invention

According to the present invention, an exopolysaccharide (EPS) production method with improved EPS productivity, EPS produced by the production method, a method for producing a fermented product comprising EPS, the method having improved EPS productivity, a fermented product produced by the production method, an agent for promoting production of EPS of bacteria of the genus Bifidobacterium, a method for promoting production of an exopolysaccharide (EPS) of a bacterium of the genus Bifidobacterium, and the like can be provided.

### BRIEF DESCRIPTION OF DRAWINGS

FIG. 1 is a diagram showing the results of a phylogenetic analysis performed by the present inventors on the nucleotide sequence of the 16S rRNA gene of the AT-APC-FucE1 strain isolated from a fecal sample originating from a human infant.

FIG. 2 is a diagram showing results obtained by observing the morphology of the AT-APC-FucE1 strain using an optical microscope (oil immersion lens, magnification of ×1000).

FIG. 3 is a diagram showing results obtained by observing the morphology of the AT-APC-FucE1 strain using an electronic microscope (magnification of ×25000).

FIG. 4 is a diagram showing results obtained by observing, with a transmission electron microscope, the AT-APC-FucE1 strain cultured in a 1% Fuc mMRS culture medium.

FIG. 5 is a diagram showing results obtained by observing, with a transmission electron microscope, the AT-APC-FucE1 strain cultured in a 1% Glu mMRS culture medium.

FIG. 6 is a diagram showing results obtained by observing, with a scanning electron microscope, EPS extracted from a culture supernatant of AT-APC-FucE1.

FIG. 7 includes diagrams showing results obtained by observing, with a scanning electron microscope (magnification of ×25000), bacterial cells in a culture solution of the Bifidobacterium animalis subsp. lactis JCM10602 strain. FIG. 7a represents the results obtained when the JCM10602 strain was cultured in a 0.5% Fuc mMRS culture medium, and FIG. 7b represents the results obtained when the JCM10602 strain was cultured in a 0.5% Glu mMRS culture medium.

### DESCRIPTION OF EMBODIMENTS

The present invention includes embodiments such as,
[1] A method for producing an exopolysaccharide (EPS), the method comprising a step A of culturing a bacterium of the genus Bifidobacterium in a culture medium comprising L-fucose (hereinafter also referred to as the “EPS production method of the present invention”);
[2] an EPS produced by the EPS production method of the present invention (hereinafter also referred to as the “EPS of the present invention”);
[3] a method for producing a fermented product comprising EPS, the method comprising a step a of culturing a bacterium of the genus Bifidobacterium in a fermented product raw material comprising L-fucose (hereinafter, also referred to as the “fermented product production method of the present invention”);
[4] a fermented product produced by the fermented product production method of the present invention (hereinafter, the fermented product thereof is also referred to as the “fermented product of the present invention”);
[5] an agent for promoting production of EPS of bacteria of the genus Bifidobacterium (hereinafter, also referred to as the “agent for promoting production of EPS of the present invention”), the agent comprising L-fucose as an active ingredient; and
[6] a method for promoting production of an exopolysaccharide (EPS) of a bacterium of the genus Bifidobacterium, the method comprising a step A of culturing the bacterium of the genus Bifidobacterium in a culture medium comprising L-fucose (hereinafter, also referred to as the “EPS production promotion method of the present invention”).

### Bacterium of Genus Bifidobacterium

The “bacterium of the genus Bifidobacterium” in the present specification is not particularly limited as long as the bacterium is a bacterium of the genus Bifidobacterium having an ability to produce EPS, and a preferable example includes a bacterium of the genus Bifidobacterium having an ability to assimilate L-fucose.

The bacterium of the genus Bifidobacterium is not particularly limited as long it belongs to the genus Bifidobacterium, and examples thereof include one or more species selected from the group consisting of Bifidobacterium breve, Bifidobacterium bifidum, Bifidobacterium longum subsp. longum, Bifidobacterium animalis subsp. animalis (hereinafter, also referred to as “Bifidobacterium animalis”), Bifidobacterium animalis subsp. lactis (hereinafter, also referred to as “Bifidobacterium lactis”), Bifidobacterium longum subsp. infantis, Bifidobacterium adolescentis, Bifidobacterium angulatum, Bifidobacterium catenulatum, and Bifidobacterium pseudocatenulatum, and among these, the bacterium of the genus Bifidobacterium is preferably one or more species selected from the group consisting of Bifidobacterium breve, Bifidobacterium lactis, Bifidobacterium animalis, and Bifidobacterium longum subsp. infantis, and among these, the bacterium of the genus Bifidobacterium is more preferably Bifidobacterium breve, Bifidobacterium lactis, or Bifidobacterium animalis, and is even more preferably Bifidobacterium breve or Bifidobacterium lactis.

The bacterium of the genus Bifidobacterium may be, for example, a bacterium isolated from nature such as from an infant fecal sample, or a bacterium obtained from a culture collection.

The above-mentioned “bacterium of the genus Bifidobacterium having an ability to produce EPS” means a bacterium of the genus Bifidobacterium that can produce EPS when cultured under conditions suitable for the bacterium of the genus Bifidobacterium. Examples of the “bacterium of the genus Bifidobacterium having an ability to produce EPS” include, for example, bacteria of the genus Bifidobacterium for which EPS production can be confirmed when anaerobically cultured at 37° C. for 12 hours or longer (for example, 96 hours) in an mMRS liquid culture medium comprising from 0.5 to 2 wt. % (or 1 wt. %) of D-glucose, and preferable examples include a bacterium of the genus Bifidobacterium from which EPS of an amount of 0.5 mg/1000 mL or more can be obtained when EPS is extracted with trichloroacetic acid from the culture supernatant by a method of culturing anaerobically at 37° C. for 96 hours in a 0.5% Glu mMRS culture medium according to the method described in Test 5 of the Examples. In addition, the amount of EPS (preferably, the amount of EPS extracted from the culture supernatant with trichloroacetic acid) produced by the above-described culturing (for example, culturing anaerobically at 37° C. for 12 hours or 96 hours in an mMRS liquid culture medium comprising 0.5 or 1 wt. % of D-glucose) can be evaluated as the EPS production ability of the bacterium of the genus Bifidobacterium.

Specific examples of the above-mentioned “bacterium of the genus Bifidobacterium having an ability to assimilate L-fucose” include bacterium of the genus Bifidobacterium that can grow (preferably, can proliferate) when anaerobically cultured at 37° C. in a mMRS liquid culture medium comprising from 0.5 to 2 wt. % of L-fucose. More specific examples include bacteria of the genus Bifidobacterium having a value of 0.1 or greater (preferably 0.2 or greater, more preferably 0.3 or greater, even more preferably 0.4 or greater, yet even more preferably 0.5 or greater, and still even more preferably 0.6 or greater), the value thereof being obtained by inoculating, at 1 v/v %, bacterium of the genus Bifidobacterium into 1 mL of an mMRS liquid culture medium comprising 0.5 or 1 wt. % of L-fucose, culturing anaerobically at 37° C. for 48 hours, and then measuring the turbidity (OD600) of the culture solution and subtracting, from the measured value of the turbidity thereof, the turbidity (OD600) of a control culture solution (a culture solution obtained by culturing, in the same manner, a culture medium not inoculated with the bacterium of the genus Bifidobacterium). In the present specification, the term “L-fucose assimilation capability” in quantitative comparison of the ability to assimilate L-fucose means a value obtained by inoculating, at 1 v/v %, a bacterium of the genus Bifidobacterium into 1 mL of an mMRS liquid culture medium comprising 0.5 or 1 wt. % of L-fucose, culturing anaerobically at 37° C. for 48 hours, and then measuring the turbidity (OD600) of the culture solution and subtracting, from the measured value of the turbidity thereof, the turbidity (OD600) of a control culture solution (a culture solution obtained by culturing, in the same manner, a culture medium not inoculated with the bacterium of the genus Bifidobacterium).

Examples of bacterium of the genus Bifidobacterium that can be suitably used in the present invention include bacterium of the genus Bifidobacterium for which the EPS productivity through the bacterium of the genus Bifidobacterium is improved by culturing in a culture medium comprising L-fucose, and specific preferred examples thereof include the Bifidobacterium breve AT-APC-FucE1 strain and Bifidobacterium lactis (for example, the JCM10602 strain). The degree to which the productivity of EPS by the bacterium of the genus Bifidobacterium is improved by culturing in a culture medium comprising L-fucose is not particularly limited.

Note that the abovementioned “bacterium of the genus Bifidobacterium for which the productivity of EPS through the bacterium of the genus Bifidobacterium is improved by culturing in a culture medium comprising L-fucose” means bacterium of the genus Bifidobacterium for which the EPS productivity is improved when cultured in a culture medium comprising L-fucose compared to when cultured in a culture medium not comprising L-fucose under the same culturing conditions with the exception of the culture medium that is used. Examples of such a bacterium include a bacterium of the genus Bifidobacterium for which the EPS productivity is improved when cultured in a culture medium obtained by adding L-fucose to a culture medium not comprising L-fucose compared to when cultured in the culture medium not comprising L-fucose, and a bacterium of the genus Bifidobacterium for which, in comparison to a case in which the bacterium is cultured in a culture medium comprising glucose and not comprising L-fucose (for example, an mMRS liquid culture medium comprising D-glucose), the EPS productivity is improved when cultured in a culture medium in which the same concentration of L-fucose is used in place of glucose in the culture medium (to thereby obtain an mMRS liquid culture medium comprising L-fucose), and a more specific example is a bacterium of the genus Bifidobacterium for which the ability to produce EPS is improved 10 wt. % or more, and preferably 20 wt. % or more.

The Bifidobacterium breve AT-APC-FucE1 strain is a strain isolated by the present inventors as a strain that is capable of assimilating fucose, the strain being isolated from a fecal sample originating from a human infant. The nucleotide sequence (SEQ ID NO: 1) of the 16S rRNA gene of the AT-APC-FucE1 strain is 99.9% identical in terms of sequence to the nucleotide sequence of the 16S rRNA gene of the Bifidobacterium breve standard strain. Based on a phylogenetic analysis of the nucleotide sequences of the 16S rRNA genes described in the Examples below, the AT-APC-FucE1 strain is considered to be the Bifidobacterium breve. Regarding this novel AT-APC-FucE1 strain, on Sep. 15, 2021 (date of deposit), the present inventors submitted an application for deposit of the novel strain as a Bifidobacterium breve AT-APC-FucE1 strain to the National Institute of Technology and Evaluation (NITE) Patent Microorganisms Depositary (NPMD) (address: 2-5-8 Kazusakamatari, Kisarazu City, Chiba Prefecture, 292-0818, Japan) (Receipt No. NITE AP-03537), but the new strain was not accepted.

Therefore, the present inventors submitted an application for deposit of the same strain to another depository. That is, the novel AT-APC-FucE1 strain was deposited on Jun. 7, 2022 (date of deposit) at the German Collection of Microorganisms and Cell Cultures (DSMZ) (address: Inhoffenstr. 7B, D-38124 Braunschweig, Germany) as the Bifidobacterium breve AT-APC-FucE1 strain (DSMZ deposit number; DSM 34284).

### Microbiological Properties of AT-APC-FucE1 Strain

Small white non-transparent colonies are formed on an MRS agar medium. When Gram staining is implemented, Gram positive results are obtained, and a branched morphology typical of bifidobacteria is exhibited. The bacterium grows only under anaerobic conditions and reaches a maximum turbidity level in approximately 48 hours under conditions of glucose assimilation. The Bifidobacterium breve standard strain JCM1192 does not assimilate fucose, whereas the AT-APC-FucE1 strain can assimilate fucose.

The sequence of SEQ ID NO: 1 is as follows.

GGCTACCTTGTTACGACTTAGTCCCAATCACGAGCCTCACCTTAGACGG
CTCCCTCCCGCAAGGGGTTAGGCCACCGGCTTCGGGTGCTGCCCACTTT
CATGACTTGACGGGCGGTGTGTACAAGGCCCGGGAACGCATTCACCGCG
ACGTTGCTGATTCGCGATTACTAGCGACTCCGCCTTCACGCAGTCGAGT
TGCAGACTGCGATCCGAACTGAGACCGGTTTTCAGGGATCCGCTCCAGC
TCGCGCTGTCGCATCCCGTTGTACCGGCCATTGTAGCATGCGTGAAGCC
CTGGACGTAAGGGGCATGATGATCTGACGTCATCCCCACCTTCCTCCGA
GTTAACCCCGGCGGTCCCCCGTGAGTTCCCGGCACAATCCGCTGGCAAC
ACGGGGCGAGGGTTGCGCTCGTTGCGGGACTTAACCCAACATCTCACGA
CACGAGCTGACGACGACCATGCACCACCTGTGAACCCGCCCCGAAGGGA
AACCCCATCTCTGGGATCGTCGGGAACATGTCAAGCCCAGGTAAGGTTC
TTCGCGTTGCATCGAATTAATCCGCATGCTCCGCCGCTTGTGCGGGCCC
CCGTCAATTTCTTTGAGTTTTAGCCTTGCGGCCGTACTCCCCAGGCGGG
ATGCTTAACGCGTTAGCTCCGACACGGAACCCGTGGAACGGGCCCCACA
TCCAGCATCCACCGTTTACGGCGTGGACTACCAGGGTATCTAATCCTGT
TCGCTCCCCACGCTTTCGCTCCTCAGCGTCAGTAACGGCCCAGAGACCT
GCCTTCGCCATTGGTGTTCTTCCCGATATCTACACATTCCACCGTTACA
CCGGGAATTCCAGTCTCCCCTACCGCACTCAAGCCCGCCCGTACCCGGC
GCGGATCCACCGTTAAGCGATGGACTTTCACACCGGACGCGACGAACCG
CCTACGAGCCCTTTACGCCCAATAATTCCGGATAACGCTTGCACCCTAC
GTATTACCGCGGCTGCTGGCACGTAGTTAGCCGGTGCTTATTCGAAAGG
TACACTCAACACAAAATGCCTTGCTCCCTAACAAAAGAGGTTTACAACC
CGAAGGCCTCCATCCCTCACGCGGCGTCGCTGCATCAGGCTTGCGCCCA
TTGTGCAATATTCCCCACTGCTGCCTCCCGTAGGAGTCTGGGCCGTATC
TCAGTCCCAATGTGGCCGGTCGCCCTCTCAGGCCGGCTACCCGTCGAAG
CCATGGTGGGCCGTTACCCCGCCATCAAGCTGATAGGACGCGACCCCAT
CCCATGCCGCAAAGGCTTTCCCAGAAGACCATGCGATCAACTGGAGCAT
CCGGCATTACCACCCGTTTCCAGGAGCTATTCCGGTGCATGGGGCAGGT
CGGTCACGCATTACTCACCCGTTCGCCACTCTCACCACCAAGCAAAGCC
CGATGGATCCCGTTCGACTTGCATGTGTTAAGCACGCCGCCAGCGTTCA
TC

The term “Bifidobacterium breve AT-APC-FucE1 strain” as used herein is not limited to the strain deposited under the name DSM 34284 (hereinafter, also referred to as the “deposited strain” for convenience) as described above, but also includes strains substantially isogenic (hereinafter, also referred to as an “isogenic strain”) to the deposited strain and strains substantially equivalent (hereinafter, also referred to as a “derivative strain” or a “derived strain”) to the deposited strain.

An isogenic strain of the deposited strain described above means a strain of the genus Bifidobacterium, the strain thereof being such that the nucleotide sequence of the 16S rRNA gene is identical in terms of sequence to the nucleotide sequence of the 16S rRNA gene of the deposited strain by preferably 99.5% or more (more preferably 99.8% or more, even more preferably 99.9% or more, yet even more preferably 99.95% or more, still even more preferably 99.99% or more, and even more preferably 100%), and the strain thereof having an L-fucose assimilation capability of 0.1 times or more (preferably 0.3 times or more, more preferably 0.5 times or more, even more preferably 0.7 times or more, yet even more preferably 0.9 times or more, and still even more preferably 1.0 times or more) compared to that of the deposited strain, and/or having an EPS production capability of 0.1 times or more (preferably 0.3 times or more, more preferably 0.5 times or more, even more preferably 0.7 times or more, yet even more preferably 0.9 times or more, and still even more preferably 1.0 times or more) compared to that of the deposited strain.

The above-mentioned “strain substantially equivalent to the deposited strain” means a strain that belongs to the same species as the deposited strain and can obtain an L-fucose assimilation capability of 0.8 times or more (preferably 0.9 times or more, more preferably 1.0 times or more) compared to that of the deposited strain and/or can obtain an EPS production capability of 0.8 times or more (preferably 0.9 times or more, more preferably 1.0 times or more) compared to that of the deposited strain. A strain substantially equivalent to the deposited strain may be, for example, a derivative strain whose parent strain is the deposited strain. Examples of derivative strains include strains bred from the deposited strain and strains naturally occurring from the deposited strain.

Examples of substantially equivalent strains (such as derivative strains) include the following strains.
(1) Strains determined to be identical strains by the Randomly Amplified Polymorphic DNA (RAPD) method and the Pulsed-field gel electrophoresis (PFGE) method (described on page 43 of Probiotics in food: Health and nutritional properties and guidelines for evaluation 85);
(2) strains having only genes derived from the deposited strain, having no foreign genes, and having a DNA sameness of 95% or more (preferably 98% or more); and
(3) strains bred from the deposited strain (with genetically engineered modifications, spontaneous mutations, and natural spontaneous mutations) and having the same traits.

In addition, in the present invention, when culturing bacteria of the genus Bifidobacterium, bacteria of the genus Bifidobacterium alone may be used, or microorganisms other than bacteria of the genus Bifidobacterium may be used in combination with the bacteria of the genus Bifidobacterium as long as growth of bacteria of the genus Bifidobacterium is not excessively inhibited. Examples of microorganisms other than bacteria of the genus Bifidobacterium include yeast and bacteria other than bacteria of the genus Bifidobacterium, such as lactic acid bacteria and bacteria of the genus Bacillus.

Examples of the above-mentioned “lactic acid bacteria” include bacteria that produce a large amount of lactic acid (preferably lactic acid of 50% or more of consumed sugar) by lactic acid fermentation of sugar, and also include bacteria of the genus Lactobacillus, bacteria of the genus Streptococcus, bacteria of the genus Lactococcus, bacteria of the genus Leuconostoc, bacteria of the genus Pediococcus, and bacteria of the genus Enterococcus.

### Culture Medium Comprising L-Fucose

The “culture medium comprising L-fucose” in the present specification may be a culture medium generally used for bacteria culturing, with the exception that the culture medium comprises L-fucose. Examples of the “culture medium comprising L-fucose” include culture mediums comprising a carbon source (only L-fucose, or L-fucose and another carbon source) and a nitrogen source, and the culture medium comprising L-fucose may further comprise other components.

The L-fucose may be extracted from seaweed such as kelp and wakame seaweed by a known method, or a commercially available product may be used.

The concentration of L-fucose in the “culture medium comprising L-fucose” is not particularly limited as long as the effects of the present invention can be obtained, and the concentration thereof may be from 2 to 100 g/L, and is preferably from 5 to 90 g/L, from 10 to 80 g/L, or the like.

The carbon source in the “culture medium comprising L-fucose” is preferably L-fucose alone from the viewpoint of further improving the EPS productivity, but a carbon source other than L-fucose may be used in combination. Examples of the “carbon source other than L-fucose” include carbon sources that are assimilable by the used bacterium of the genus Bifidobacterium. A single type of “carbon source other than L-fucose” may be used, or a combination of two or more types thereof may be used. Specific examples of the “carbon source other than L-fucose” include one or more types of carbon sources selected from saccharides such as glucose, lactose, sucrose, maltose, mannose, galactose, fructose, starch hydrolysates, and molasses; sugar alcohols such as mannitol and erythritol; alcohols such as glycerol, ethanol and propanol; and organic acids such as acetic acid, malic acid, lactic acid, citric acid, tartaric acid, succinic acid, fumaric acid, propionic acid, and malonic acid, and of these, preferable examples include one or more types selected from the group consisting of glucose, lactose, sucrose, maltose, galactose, and fructose.

When the culture medium comprising L-fucose comprises a carbon source other than L-fucose, the concentration of the carbon source other than L-fucose in the culture medium is not particularly limited as long as the effects of the present invention can be obtained, and examples of the concentration thereof include from greater than 0 g/L to 100 g/L or from greater than 0 g/L to 50 g/L, but from the viewpoint of further improving the EPS productivity, preferable examples of the concentration thereof include from greater than 0 g/L to 30 g/L, from greater than 0 g/L to 15 g/L, and from greater than 0 g/L to 8 g/L.

Examples of the “nitrogen source” in the present specification include a nitrogen source that are assimilable by the used bacteria of the genus Bifidobacterium. A single type of nitrogen source may be used, or a combination of two or more types thereof may be used. Specific examples of the nitrogen source contained in the L-fucose-comprising culture medium used in the present invention include one or more selected from amino acids, potassium nitrate, ammonium citrate, ammonium nitrate, ammonium sulfate, ammonium phosphate, ammonium chloride, ammonia, urea, casein, polypeptone, peptone, casamino acid, NZ amine, tryptose, corn steep liquor, yeast extract, meat extract, and fish extract, and among these, preferable examples include one or more types selected from amino acid, potassium nitrate, ammonium citrate, ammonium nitrate, ammonium sulfate, ammonium phosphate, ammonium chloride, yeast extract, meat extract, and fish extract.

The nitrogen source concentration in the culture medium comprising L-fucose is not particularly limited as long as the effects of the present invention can be obtained, and examples of the concentration thereof include from 1 to 30 g/L and from 1 to 20 g/L.

In the present specification, the “other components” in the culture medium or fermented product raw material are not particularly limited as long as they do not excessively inhibit the growth of the bacteria of the genus Bifidobacterium when added to the culture medium or the fermented product raw material, and adding inorganic ions or vitamins as necessary is effective. Examples of the inorganic ions include potassium ions, sodium ions, calcium ions, magnesium ions, iron ions, manganese ions, molybdenum ions, phosphate ions, chloride ions, and sulfate ions. Examples of vitamins include thiamine, inositol, pantothenic acid, and nicotinamide.

In addition, with regard to the “culture medium comprising L-fucose”, as the culture medium component other than L-fucose, for example, an mMRS culture medium (a culture medium obtained by removing sugar components from an MRS culture medium), or a culture medium to which a carbon source such as a sugar component has been added to an mMRS culture medium can be suitably used. The composition of the mMRS culture medium is as follows.

Composition of mMRS Culture Medium

The composition of the mMRS culture medium includes 10 g/L of beef extract, 5 g/L of yeast extract, 2 g/L of ammonium citrate, 5 g/L of sodium acetate, 0.1 g/L of magnesium sulfate. 0.05 g/L of manganese sulfate, 2 g/L of dipotassium hydrogen phosphate, and 0.5 g/L of L-cysteine hydrochloride.

The pH of the “culture medium comprising L-fucose” as used herein is not particularly limited as long as the bacteria of the genus Bifidobacterium can grow, and the pH thereof may be, for example, from 4 to 9.

In the present specification, the “culture medium comprising L-fucose” may be a liquid culture medium or a plate culture medium. In the case of a plate culture medium, the gel component to be contained is not particularly limited as long as the gel component does not inhibit the growth of bacteria of the genus Bifidobacterium, but agar is preferable because it is widely used and is excellent in handling properties.

### Step A of Culturing Bacterium of Genus Bifidobacterium in Culture Medium Comprising L-Fucose

The step A of culturing a bacterium of the genus Bifidobacterium in a culture medium comprising L-fucose is not particularly limited as long as step A is a step of culturing a bacterium of the genus Bifidobacterium in a culture medium comprising L-fucose.

The culturing conditions when culturing the bacterium of the genus Bifidobacterium in a culture medium comprising L-fucose are not particularly limited as long as the effects of the present invention can be obtained.

The culturing temperature is not particularly limited as long as the culturing temperature is a temperature at which the bacterium of the genus Bifidobacterium can grow, and is, for example, from 15 to 50° C., preferably from 30 to 50° C., and more preferably from 35 to 45° C.

The culturing time is not particularly limited and may be from 6 hours to 5 days, but from the viewpoints of proliferation of bacteria of the genus Bifidobacterium and the EPS produced, the culturing time is preferably from 12 to 48 hours, and particularly preferably from 12 to 24 hours.

Culturing of the bacterium of the genus Bifidobacterium is preferably carried out under anaerobic conditions, and for example, the culturing can be carried out while circulating an anaerobic gas such as carbon dioxide. Alternatively, the culturing may be carried out under micro-aerobic conditions such as liquid static culturing.

### Optional Step

In the EPS production method of the present invention, the cultured product of the EPS obtained by the culturing in step A may be used as is, but the EPS production method of the present invention may further include a step B of collecting the EPS from the cultured product obtained by culturing in step A.

A known method can be used as the method for collecting EPS from the cultured product.

An example of a method for collecting only acidic polysaccharides (acidic EPS) is a method comprising the following steps 1 to 5.

1. Remove bacterial cells from the cultured product by centrifugation.

2. Add trichloroacetic acid such that the final concentration is around 5 to 10 wt. %, precipitate the proteins, and then subject to centrifugation.

3. Recover high molecular weight polysaccharides and proteins as precipitates through ethanol precipitation.

4. Remove the proteins and nucleic acids.

a) Decompose the nucleic acids using DNase and RNase.

b) Degrade the proteins using proteinase.

c) Thermally denature the proteins, and then subject to centrifugation and dialysis.

5. After the acidic polysaccharides are adsorbed by an anion exchange resin, elute and recover the acid polysaccharides.

An example of a method for collecting only neutral polysaccharides (neutral EPS) is a method comprising the following steps 1 to 5.

1. Add trichloroacetic acid to the culture medium such that the final concentration is 10 wt. %, and denature the proteins.

2. Remove the denatured proteins and bacterial cells from the cultured product by centrifugation.

3. Precipitate and recover high molecular weight polysaccharides through ethanol precipitation.

4. Adsorb acidic polysaccharides using an anion exchange resin, and recover neutral polysaccharides from the remaining eluate.

5. Decompose the nucleic acids through DNase and RNase treatments.

6. Degrade proteins through a proteinase treatment.

7. Heat for 10 minutes at 90° C. to deactivate the enzymes.

8. Purify the neutral polysaccharides through ethanol precipitation and dialysis.

### EPS Produced by EPS Production Method of the Present Invention

The EPS of the present invention is not particularly limited as long as it is an EPS produced by the EPS production method of the present invention.

### Fermented Product Raw Material Comprising L-Fucose

In the present specification, the “fermented product raw material comprising L-fucose” is not particularly limited as long as it is a fermented product raw material comprising L-fucose.

The concentration of L-fucose in the “fermented product raw material comprising L-fucose” is not particularly limited as long as the effects of the present invention can be obtained, and the concentration thereof is, for example, from 2 to 100 g/L, and preferably from 5 to 90 g/L, from 10 to 80 g/L, or the like.

The “fermented product raw material” is not particularly limited as long as it is a raw material for a fermented product, and such a fermented product raw material may comprise a carbon source and a nitrogen source. The fermented product raw material may further comprise the above-described “other components”.

The “fermented product raw material” may be in a solid form or a liquid form.

Specific preferred examples of the “fermented product raw material” include raw materials for fermented dairy products and raw materials for fermented soybean products. Examples of the fermented dairy product raw material include a raw material comprising a milk raw material, and examples of the fermented soybean product raw material include steamed soybeans.

In the present specification, examples of the “milk raw material” typically include “milk” as defined by the Japan Ministerial Ordinance on Milk and Milk Products, that is, milk such as raw milk, cow's milk, special cow's milk, raw goat's milk, pasteurized goat's milk, raw sheep's milk, composition-modified cow's milk, low-fat cow's milk, non-fat cow's milk, and processed milk, or a composition comprising a non-fat milk solid content equal to or higher than these (that is, 8% or higher), but the milk raw material is not particularly limited as long as it is a composition comprising a milk component. Examples of the “milk component” in the present specification include one or more types selected from the group consisting of milk fat derived from the “milk” defined by the Japan Ministerial Ordinance on Milk and Milk Products, and non-fat milk solids derived from the “milk” thereof (for example, proteins derived from the “milk” and/or sugars derived from the “milk”).

The “milk raw material” in the present specification can be prepared using milk, a dairy product, or the like. When milk and/or dairy products are used, the “milk raw material” can more specifically be prepared using one or more types selected from the group consisting of cow's milk, water buffalo milk, sheep's milk, goat's milk, horse's milk, concentrated milk, skim milk, concentrated skim milk, skim milk powder, partially skim milk powder, whole milk powder, cream, butter, buttermilk, condensed milk, lactose, milk protein concentrate, whey protein concentrate, and water.

In the present specification, examples of the “fermented dairy product raw material” include those having a concentration of milk component solids of from 1 to 16 wt. %, preferably from 2 to 14 wt. %, and more preferably from 4 to 12 wt. %, and/or those having a concentration of non-fat milk solids of from 1 to 18 wt. %, preferably from 2 to 16 wt. %, more preferably from 2 to 14 wt. %, even more preferably from 4 to 12 wt. %, from 6 to 10 wt. % or from 7 to 9 wt. %, and/or those having a concentration of milk fat of from 0 to 8 wt. %, preferably from 0.1 to 7 wt. %, and more preferably from 0.5 to 4 wt. % or from 1 to 3 wt. %.

### Step a of Culturing Bacterium of Genus Bifidobacterium in Fermented Product Raw Material Comprising L-Fucose

The step a of culturing a bacterium of the genus Bifidobacterium in a fermented product raw material comprising L-fucose is not particularly limited as long as the step a is a step of culturing a bacterium of the genus Bifidobacterium in a fermented product raw material comprising L-fucose.

The culturing conditions when culturing the bacterium of the genus Bifidobacterium in the fermented product raw material comprising L-fucose are not particularly limited as long as the effects of the present invention can be obtained, and examples of the culturing temperature, the culturing time, the oxygen conditions, and the like include the conditions and preferable conditions exemplified with regard to step A.

### Fermented Product

In the present specification, the “fermented product” is not particularly limited as long as it is a fermented product produced by the fermented product production method of the present invention. Examples of the “fermented product” include fermented milk and fermented soybeans.

The term “fermented product” as used herein includes EPS produced by the bacterium of the genus Bifidobacterium according to the present invention. The concentration of EPS comprised in the “fermented product” is not particularly limited, and for example, the EPS concentration may be from 0.001 to 10 wt. %, from 0.01 to 10 wt. %, or from 0.01 to 5 wt. %.

In addition, the “fermented product” herein may comprise L-fucose. The concentration of L-fucose comprised in the “fermented product” is not particularly limited, and for example, the L-fucose concentration may be from 0.001 to 5 wt. %, from 0.01 to 5 wt. %, from 0.05 to 5 wt. %, from 0.05 to 3 wt. %, or from 0.05 to 2 wt. %.

### Agent for Promoting Production of EPS

The “Agent for promoting production of EPS” of the present invention is not particularly limited as long as it comprises L-fucose as an active ingredient. Such an agent for promoting production of EPS can be used by, for example, adding the EPS production promoter to a culture medium such as a general culture medium that is used when culturing bacteria of the genus Bifidobacterium.

The “Agent for promoting production of EPS” of the present invention may be in a solid form or a liquid form, but is preferably in a solid form from the viewpoint of storage properties. When the agent for promoting production of EPS is in a solid form, the agent for promoting production of EPS is preferably in the form of a powder or granules from the viewpoint of solubility in the culture medium. When the agent for promoting production of EPS is in a liquid form, the agent for promoting production of EPS comprises a liquid carrier in addition to L-fucose, and examples of the liquid carrier include water.

The concentration of L-fucose in the “Agent for promoting production of EPS” of the present invention is not particularly limited, and the “Agent for promoting production of EPS” may consist of only L-fucose. However, examples of other concentrations of the L-fucose therein include from 0.1 to 95 wt. %, from 0.5 to 90 wt. %, from 1 to 90 wt. %, from 3 to 85 wt. %, and from 5 to 80 wt. %.

When the agent for promoting production of EPS is in a solid form, the EPS production promoter may further comprise optional components in addition to the L-fucose, and when the agent for promoting production of EPS is in a liquid form, the agent for promoting production of EPS may further comprise optional components in addition to the L-fucose and liquid carrier. Examples of such optional components include some or all of the culture medium components other than L-fucose as described with regard to the “culture medium comprising L-fucose” in the present specification. When the agent for promoting production of EPS comprises some or all of the culture medium components other than L-fucose as described with regard to the “culture medium comprising L-fucose” in the present specification, the agent for promoting production of EPS can also be referred to as a culture medium for promoting EPS production.

### EPS Production Promotion Method

The “EPS production promotion method” of the present invention is not particularly limited as long as it is a method for promoting production of exopolysaccharides (EPS) by a bacterium of the genus Bifidobacterium and includes the step A of culturing the bacterium of the genus Bifidobacterium in a culture medium comprising L-fucose.

The step A is as described above.

Hereinafter, the present invention will be described more specifically through examples, but the present invention is not limited by these examples.

### EXAMPLES

### [Test 1] Isolation of Bacterium of Genus Bifidobacterium Capable of Assimilating L-Fucose

An mMRS liquid culture medium comprising 1 wt. % of L-fucose (hereinafter, also referred to as a “1% Fuc mMRS culture medium”) (composition: 10 g/L of beef extract, 5 g/L of yeast extract, 2 g/L of ammonium citrate, 5 g/L of sodium acetate, 0.1 g/L of magnesium sulfate, 0.05 g/L of manganese sulfate, 2 g/L of dipotassium hydrogen phosphate, 0.5 g/L of L-cysteine hydrochloride, and 1% L-fucose) was prepared.

The 1% Fuc mMRS culture medium was inoculated with a predetermined amount (an amount of 1 wt. % of the culture medium) of a fecal sample derived from a plurality of human infants, after which enrichment culturing and subculturing were repeated under anaerobic conditions (CO₂) at 37° C., and a bacterial strain capable of assimilating L-fucose (the bacterial strain thereof is also referred to as the “AT-APC-FucE1 strain”) was isolated.

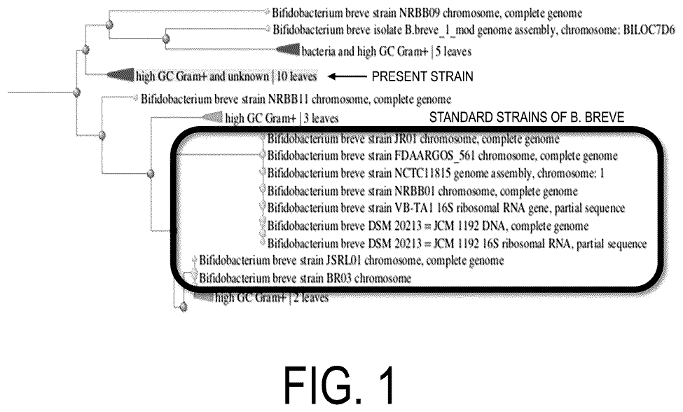

The nucleotide sequence of the 16S rRNA gene of the AT-APC-FucE1 strain was identified by sequencing. The nucleotide sequence of the 16S rRNA gene is indicated by SEQ ID NO: 1. The nucleotide sequence of the 16S rRNA gene of the AT-APC-FucE1 strain was confirmed to be 99% identical in terms of sequence to the nucleotide sequence of the 16S rRNA gene of the standard strain of Bifidobacterium breve. When the nucleotide sequence of the 16S rRNA gene was subjected to phylogenetic analysis with various Bifidobacterium breve strains (FIG. 1), the AT-APC-FucE1 strain was most closely related to Bifidobacterium breve and was considered to be Bifidobacterium breve. On Jun. 7, 2022 (date of deposit), the present inventors deposited the AT-APC-FucE1 strain with the German Collection of Microorganisms and Cell Cultures GmbH (DSMZ) (DSMZ deposit number; DSM 34284).

The present inventors examined the microbiological properties of the AT-APC-FucE1 strain and found the following.

The strain formed small white non-transparent colonies on an MRS agar culture medium. Gram staining was then implemented, Gram positive results were obtained, and a branched morphology typical of bifidobacteria was exhibited. The strain grew only under anaerobic conditions and reached a maximum turbidity level at approximately 48 hours under conditions of glucose assimilation.

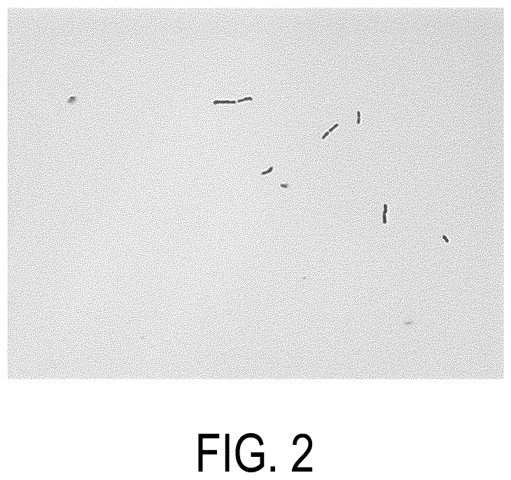

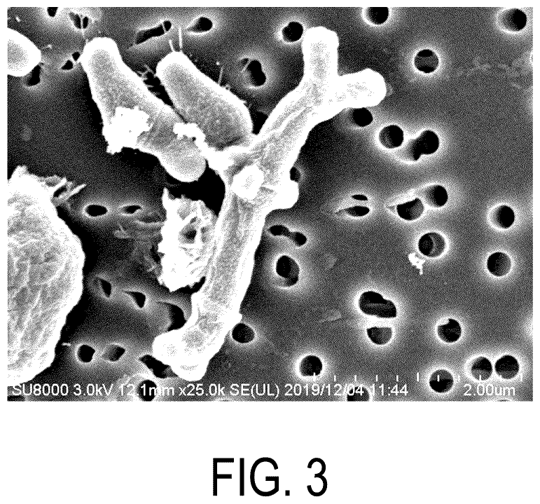

FIGS. 2 and 3 show the results of observation of the morphology of the AT-APC-FucE1 strain using an optical microscope and a scanning electron microscope (SEM), respectively. As is clear from FIGS. 2 and 3, the AT-APC-FucE1 strain had a length of from 1 to 2 μm and exhibited a rod-shaped or branched morphology.

### [Test 2] Confirmation of EPS Production Through Culturing of AT-APC-FucE1 Strain

An mMRS liquid culture medium comprising 0.5 wt. % and an mMRS liquid culture medium comprising 1 wt. % of D-glucose (hereinafter, the respective mMRS liquid culture mediums are also referred to as a “0.5% Glu mMRS culture medium” and a “1% Glu mMRS culture medium”, respectively) (composition: 10 g/L of beef extract, 5 g/L of yeast extract, 2 g/L of ammonium citrate, 5 g/L of sodium acetate, 0.1 g/L of magnesium sulfate, 0.05 g/L of manganese sulfate, 2 g/L of dipotassium hydrogen phosphate, 0.5 g/L of L-cysteine hydrochloride, and 0.5% or 1% D-glucose) were prepared. The 0.5% Glu mMRS culture medium was inoculated with the AT-APC-FucE1 strain, and pre-culturing was implemented anaerobically at 37° C. for 24 hours.

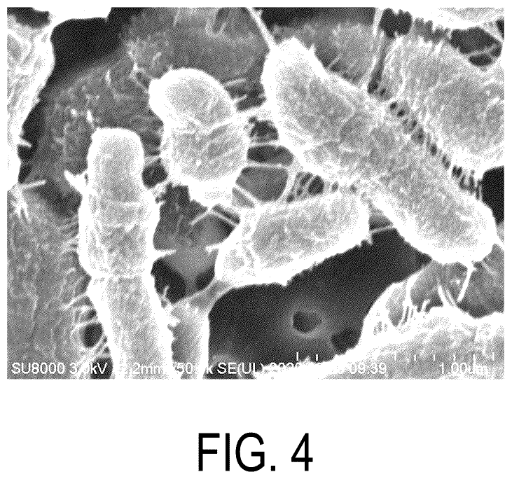

The 1% Fuc mMRS culture medium was then inoculated with 100 μL of the pre-cultured culture solution, and anaerobic culturing was carried out at 37° C. The AT-APC-FucE1 strain was collected from the culture solution that had been cultured for 12 hours, after which the bacterial cells were washed with phosphate-buffered saline (PBS), and subsequently, the bacterial cells were immobilized on a membrane, and the EPS production by the bacterial cells was confirmed using a transmission electron microscope. The results are presented in FIG. 4.

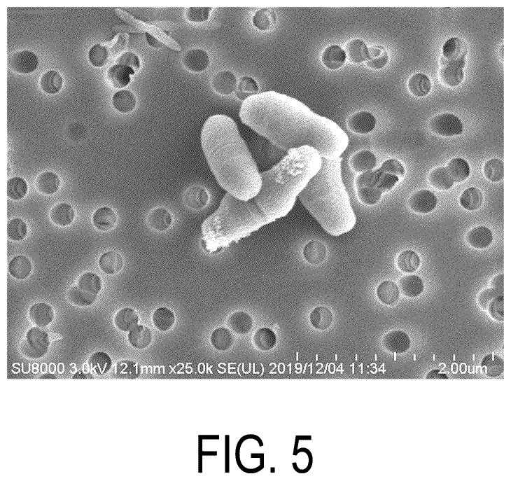

Meanwhile, 100 μL of the above-mentioned culture solution that had been pre-cultured was inoculated into a 1% Glu mMRS culture medium as a control and anaerobically cultured at 37° C. The AT-APC-FucE1 strain was collected from the culture solution that had been cultured for 12 hours, after which the bacterial cells were washed with phosphate-buffered saline (PBS), and subsequently, the bacterial cells were immobilized on a membrane, and the EPS production of the bacterial cells was confirmed using a transmission electron microscope. The results are shown in FIG. 5.

As is clear from the results shown in FIGS. 4 and 5, when the AT-APC-FucE1 strain was cultured in a culture medium comprising L-fucose, EPS production was remarkably promoted in comparison to the case in which the AT-APC-FucE1 strain was cultured in the culture medium comprising D-glucose and not comprising L-fucose.

### [Test 3] Influence of Presence or Absence of L-Fucose in Culture Medium on EPS Production Amount by AT-APC-FucE1 Strain

The EPS production amount was compared between a case in which the AT-APC-FucE1 strain was cultured in a culture medium comprising L-fucose and a case in which the AT-APC-FucE1 strain was cultured in a medium not comprising L-fucose.

The 0.5% Glu mMRS culture medium was inoculated with the AT-APC-FucE1 strain, and pre-culturing was implemented anaerobically at 37° C. for 24 hours. An amount of 100 μL of the pre-cultured culture solution was inoculated into each of a 0.5% Fuc mMRS culture medium (mMRS liquid culture medium comprising 0.5 wt. % of L-fucose) and a 0.5% Glu mMRS culture medium, and then cultured anaerobically at 37° C. for 96 hours.

The amount of crude EPS extracted from the supernatant of each culture solution was approximately the same (13.33 mg/1000 mL). However, when the amount of protein in these crude EPS samples was measured, and the amount of protein was subtracted from the amount of crude EPS, the amount of EPS produced in the culture medium that used D-glucose (that is, the 0.5% Glu mMRS culture medium) was 9.34 mg/1000 mL, whereas the amount of EPS produced in the culture medium that used L-fucose (that is, the 0.5% Fuc mMRS culture medium) was 13.14 mg/1000 mL.

From these results, it was quantitatively demonstrated that when the AT-APC-FucE1 strain was cultured in a culture medium comprising L-fucose, EPS production was remarkably promoted in comparison to the case in which the AT-APC-FucE1 strain was cultured in the culture medium comprising D-glucose and not comprising L-fucose.

[Test 4] Morphology of EPS Extracted from AT-APC-FucE1

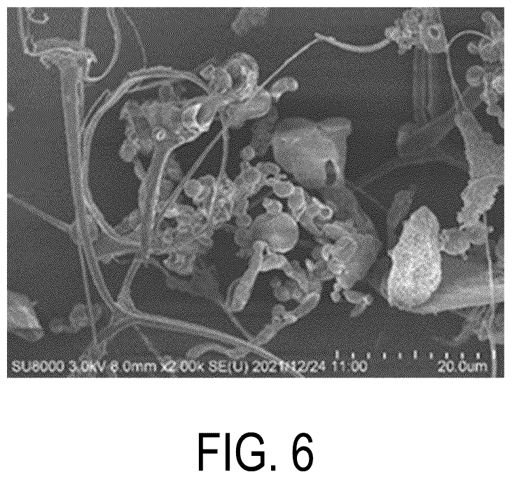

When the EPS extracted from the culture supernatant of AT-APC-FucE1 was observed with a scanning electron microscope, a morphology similar to that of the EPS observed in lactic acid bacteria and the like was confirmed (FIG. 6).

### [Test 5] Confirmation of Induction of EPS Production by L-Fucose in Other Bacteria of Genus Bifidobacterium

In order to confirm whether the production of EPS is induced by L-fucose in other bacteria of the genus Bifidobacterium, the following test was carried out using the JCM10602 strain and the JCM1192 strain.

Note that the JCM10602 strain is Bifidobacterium animalis subsp. lactis (i.e., Bifidobacterium lactis), which is frequently used as a probiotic and is known to produce EPS at a high level, and the JCM1192 strain is a standard strain of Bifidobacterium breve, which is the same species as AT-APC-FucE1.

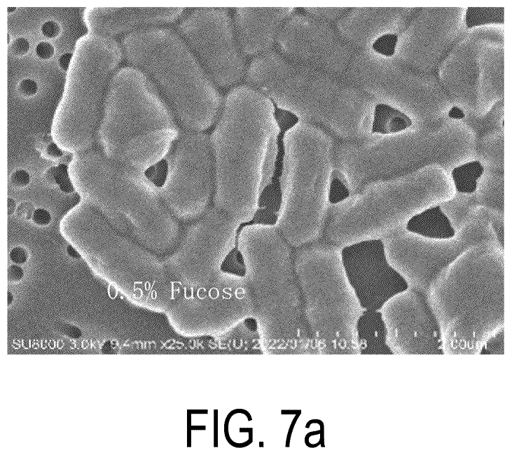

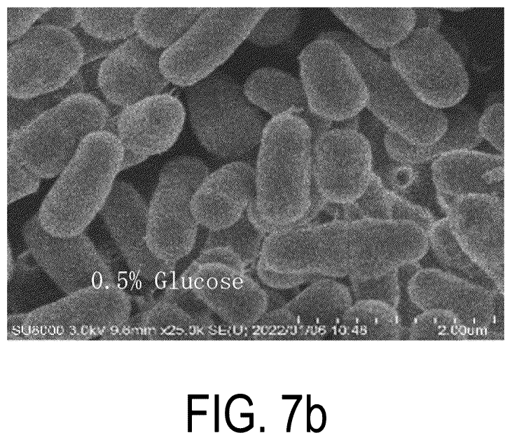

The JCM10602 strain was anaerobically cultured in a 0.5% Fuc mMRS culture medium and in a 0.5% Glu mMRS culture medium at 37° C. for 96 hours by the method described in Test 3 above. Each culture supernatant was observed with a transmission electron microscope, and the results are shown in FIG. 7. The results demonstrated that the production of EPS was improved when the bacterial cells were cultured in the 0.5% Fuc mMRS culture medium (FIG. 7a) as compared to when the bacterial cells were cultured in the 0.5% Glu mMRS culture medium (FIG. 7b).

When the EPS extracted from the culture supernatant was assayed, the amount of EPS produced in the culture medium using D-glucose (that is, the 0.5% Glu mMRS culture medium) was 132.97 mg/1000 mL (when extracted with trichloracetic acid) and 4.74 mg/1000 mL (when extracted with ethanol), whereas the amount of EPS produced in the culture medium using L-fucose (that is, the 0.5% Fuc mMRS culture medium) was 166.2 mg/1000 mL (when extracted with trichloracetic acid) and 13.33 mg/1000 mL (when extracted with ethanol). That is, it was quantitatively demonstrated that when the JCM10602 strain was cultured in a culture medium comprising L-fucose, EPS production was remarkably promoted as compared to the case in which the JCM10602 strain was cultured in a medium comprising D-glucose but not comprising L-fucose.

From these results, it was demonstrated that the induction of EPS production by L-fucose is observed not only with the AT-APC-FucE1 strain of Bifidobacterium breve, but also with other strains of bacteria that belong to the genus Bifidobacterium and have an ability to produce EPS (for example, the Bifidobacterium lactis JCM10602 strain).

Note that significant production induction by L-fucose was not confirmed with JCM1192, which is a standard strain of Bifidobacterium breve of the same species as AT-APC-FucE1.

### INDUSTRIAL APPLICABILITY

According to the present invention, an exopolysaccharide (EPS) production method with improved EPS productivity, EPS produced by the production method, a method for producing a fermented product comprising EPS, the method having improved EPS productivity, a fermented product produced by the production method, an agent for promoting production of EPS of bacteria of the genus Bifidobacterium, a method for promoting production of an exopolysaccharide (EPS) of a bacterium of the genus Bifidobacterium, and the like can be provided.

1. A method for producing an exopolysaccharide (EPS), the method comprising a step A of culturing a bacterium of the genus Bifidobacterium in a culture medium comprising L-fucose.

2. The method according to claim 1, wherein the bacterium of the genus Bifidobacterium is Bifidobacterium breve or Bifidobacterium lactis.

3. An EPS produced by the production method according to claim 1.

4. A method for producing a fermented product comprising EPS, the method comprising a step a of culturing a bacterium of the genus Bifidobacterium in a fermented product raw material comprising L-fucose.

5. The method according to claim 4, wherein the bacterium of the genus Bifidobacterium is Bifidobacterium breve or Bifidobacterium lactis.

6. The method according to claim 4, wherein the fermented product raw material is a fermented dairy product raw material, and the fermented product is a fermented dairy product.

7. A fermented product produced by the production method according to claim 4.

8. The fermented product according to claim 7, wherein the fermented product is a fermented dairy product.

9. (canceled)

10. A method for promoting production of an exopolysaccharide (EPS) of a bacterium of the genus Bifidobacterium, the method comprising a step A of culturing the bacterium of the genus Bifidobacterium in a culture medium comprising L-fucose.

11. An EPS produced by the production method according to claim 2.

12. A fermented product produced by the production method according to claim 5.

13. A fermented product produced by the production method according to claim 6.


SEQUENCE LISTING (1 sequence)

SEQ ID NO: 1 - DNA, 1472 bp, Bifidobacterium breve
  source 1..1472: mol_type = genomic DNA
ggctaccttgttacgacttagtcccaatcacgagcctcaccttagacggctccctcccgcaaggggttaggccaccggcttcgggtgctgcccactttcatgacttgacgggcggtgtgtacaaggcccgggaacgcattcaccgcgacgttgctgattcgcgattactagcgactccgccttcacgcagtcgagttgcagactgcgatccgaactgagaccggttttcagggatccgctccagctcgcgctgtcgcatcccgttgtaccggccattgtagcatgcgtgaagccctggacgtaaggggcatgatgatctgacgtcatccccaccttcctccgagttaaccccggcggtcccccgtgagttcccggcacaatccgctggcaacacggggcgagggttgcgctcgttgcgggacttaacccaacatctcacgacacgagctgacgacgaccatgcaccacctgtgaacccgccccgaagggaaaccccatctctgggatcgtcgggaacatgtcaagcccaggtaaggttcttcgcgttgcatcgaattaatccgcatgctccgccgcttgtgcgggcccccgtcaatttctttgagttttagccttgcggccgtactccccaggcgggatgcttaacgcgttagctccgacacggaacccgtggaacgggccccacatccagcatccaccgtttacggcgtggactaccagggtatctaatcctgttcgctccccacgctttcgctcctcagcgtcagtaacggcccagagacctgccttcgccattggtgttcttcccgatatctacacattccaccgttacaccgggaattccagtctcccctaccgcactcaagcccgcccgtacccggcgcggatccaccgttaagcgatggactttcacaccggacgcgacgaaccgcctacgagccctttacgcccaataattccggataacgcttgcaccctacgtattaccgcggctgctggcacgtagttagccggtgcttattcgaaaggtacactcaacacaaaatgccttgctccctaacaaaagaggtttacaacccgaaggcctccatccctcacgcggcgtcgctgcatcaggcttgcgcccattgtgcaatattccccactgctgcctcccgtaggagtctgggccgtatctcagtcccaatgtggccggtcgccctctcaggccggctacccgtcgaagccatggtgggccgttaccccgccatcaagctgataggacgcgaccccatcccatgccgcaaaggctttcccagaagaccatgcgatcaactggagcatccggcattaccacccgtttccaggagctattccggtgcatggggcaggtcggtcacgcattactcacccgttcgccactctcaccaccaagcaaagcccgatggatcccgttcgacttgcatgtgttaagcacgccgccagcgttcatc